(19) United States
(12) Patent Application Publication
(10) Pub. No.: US 2011/0046485 A1
(43) Pub. Date: Feb. 24, 2011

(54) ULTRASOUND DIAGNOSTIC IMAGING DEVICE, AND METHOD FOR GENERATING ULTRASOUND IMAGES

(75) Inventor: Kazuhito NAKATA, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: Kabushiki Kaisha Toshiba, Tokyo (JP); TOSHIBA MEDICAL SYSTEMS CORPORATION, Otawara-shi (JP)

(21) Appl. No.: 12/854,489

(22) Filed: Aug. 11, 2010

(30) Foreign Application Priority Data
Aug. 19, 2009 (JP) 2009-189863

(51) Int. Cl. A61B 8/14

(52) U.S. Cl. 600/440

(57) ABSTRACT

An ultrasound diagnostic imaging device has an optional-direction M-mode cursor set position information selector for selecting optional-direction M-mode cursor set position information, a memory, and an optional-direction M-mode cursor image generator. The memory stores optional-direction M-mode cursor set position information, associating it with the optional-direction M-mode cursor set position information selector. The optional-direction M-mode cursor image generator generates an optional-direction M-mode cursor image, based on the optional-direction M-mode cursor set position information that has been associated with the optional-direction M-mode cursor set position information selector that was selected.

### CROSS-REFERENCE TO RELATED APPLICATION

This application is based upon and claims the benefit of priority from Japanese Patent Application No. 2009-189863, filed Aug. 19, 2009; the entire contents of which are incorporated herein by reference.

### FIELD

The present embodiment relates to an ultrasound diagnostic imaging device that sets an optional-direction M-mode cursor on a B-mode diagnostic image, and a method for generating ultrasound images.

### BACKGROUND

In diagnostic imaging using an ultrasound diagnostic imaging device, the presence and the region of disease are determined by means of a B-mode image (tomographic view), which represents the region to be diagnosed, measurements are performed using M-mode images, and the severity is quantitatively evaluated. These M-mode images are images that represent the change over time of luminance levels in the direction of the scan lines in the B-mode image. In particular, M-mode images are images that represent the luminance levels of lines that run in the direction of the scan lines, with time on the horizontal axis and depth on the vertical axis.

For example, a technology to generate M-mode images using lines that run in an optional direction in the B-mode image is proposed.

When displaying M-mode images in an optional direction, an M-mode image cursor (sometimes also termed the M-mode image extraction line) is displayed so that it is overlaid on the B-mode image.

The M-mode image cursor's center position, length and angle are then set.

The setting method for the M-mode image cursor will now be described. First, a B-mode diagnostic image is displayed on the monitor for a morphological diagnosis. Next, the operator, using the input unit, sets the position (center position, length and angle) of the M-mode image cursor that is to be displayed on the B-mode diagnostic image. For example, the operator, using the input unit, in setting the M-mode image cursor at the desired position and rotating the M-mode image cursor as necessary by adjusting the center position and length of the M-mode image cursor, sets the M-mode image cursor in an optional direction with respect to the diagnostic region.

In the manner described above, the operator sets the M-mode image cursor on the B-mode diagnostic image. However, each time the scan cross-section that is scanned using ultrasound (for example, the scan cross-sections for generating images such as four-chamber view, long axis view or short axis view) is changed, it is necessary to set the center position, length and angle of the M-mode image cursor each time.

In other words, when the scan cross-section is changed, the operator, while applying an ultrasound probe to the subject with one hand, has to once again set the center position, length and angle of the M-mode image cursor. For this reason, these operations represent a burden on the operator.

### BRIEF DESCRIPTION OF THE DRAWINGS

FIG. 1 is a diagram showing an ultrasound diagnostic imaging device according to this embodiment.

FIG. 2 is a block diagram showing an image processor.

FIG. 3 is a block diagram showing a system controller.

FIG. 4 is a display example of an M-mode diagnostic image cursor selection screen.

FIG. 5 is a flow chart for describing a selection process for an optional-direction M-mode diagnostic image cursor displayed on a B-mode diagnostic image.

FIG. 6 is a flow chart for describing the selection process for the optional-direction M-mode diagnostic image cursor displayed on the B-mode diagnostic image.

FIG. 7 is a display example of a B/M-mode display screen when an optional-direction M-mode cursor image is superimposed and displayed on the B-mode diagnostic image.

FIG. 8 is a view showing a console in which buttons are installed.

FIG. 9 is a view showing a console in which a switch is installed.

FIG. 10 is a view showing a display screen in which a menu is displayed.

### DETAILED DESCRIPTION

The ultrasound diagnostic imaging device according to the present embodiment has a B-mode diagnostic image generator, an optional-direction M-mode diagnostic image generator, an optional-direction M-mode cursor set position information selector, a memory, and an optional-direction M-mode cursor image generator.

The B-mode diagnostic image generator generates B-mode diagnostic images based on echo signals obtained by scanning the subject using ultrasound.

The optional-direction M-mode diagnostic image generator generates optional-direction M-mode diagnostic images based on the set position information of the optional-direction M-mode cursor.

The optional-direction M-mode cursor set position information selector is for selecting the set position information of the optional-direction M-mode cursor.

The memory stores the set position information of the optional-direction M-mode cursor associated with the optional-direction M-mode cursor set position information selector.

The optional-direction M-mode cursor image generator generates optional-direction M-mode cursor images based on the set position information of the optional-direction M-mode cursor that has been associated with the selected optional-direction M-mode cursor set position information selector.

Below, making reference to the figures, the embodiments are described.

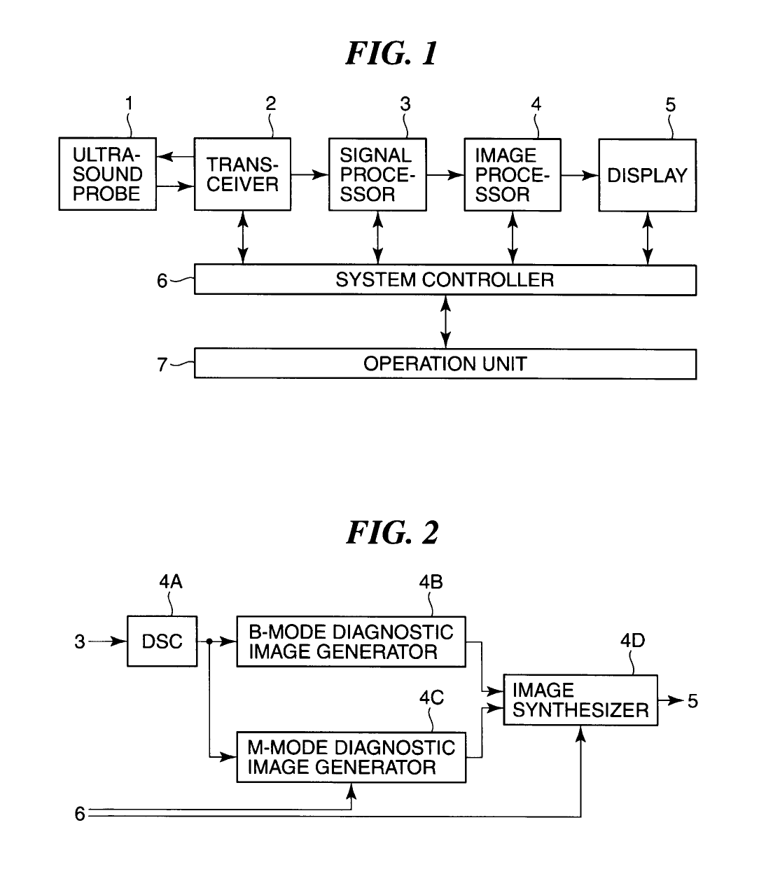

FIG. 1 is a block diagram showing an ultrasound diagnostic imaging device.

The head of an ultrasound probe 1 is brought into contact with the body surface of the subject, and the transmission and reception of ultrasound is performed. The ultrasound probe 1 has a plurality of piezoelectric vibrators. This plurality of piezoelectric vibrators is arrayed 1-dimensionally or 2-dimensionally on the head of the ultrasound probe 1. The ultrasound probe 1 receives echo signals obtained by scanning the subject using ultrasound, and outputs them to a transceiver 2.

The transceiver 2 generates ultrasound driven signals for producing ultrasound, and outputs them to the ultrasound probe 1.

Moreover, the transceiver 2 performs beamforming (delay-and-sum processing) on the ultrasound signals received over a plurality of channels obtained from the piezoelectric vibrators of the ultrasound probe 1, and outputs the echo signals obtained by the beamforming (delay-and-sum processing) to a signal processor 3.

The signal processor 3 has a B-mode processor, a Doppler processor and a color mode processor. The B-mode processor performs signal processing of the amplitude information for the echo signals output from the transceiver 2. The B-mode processor outputs to an image processor 4 the B-mode ultrasound raster data that was generated by performing the signal processing. The Doppler processor extracts the Doppler shift frequency components from the echo signals output from the transceiver 2, and by further performing FFT (Fast Fourier Transform) processing, etc., generates blood flow information.

The Doppler processor outputs the blood flow information to the image processor 4.

The color mode processor performs signal processing of the blood flow information based on the echo signals output from the transceiver 2.

The color mode processor outputs to the image processor 4 the color ultrasound raster data generated by performing the signal processing.

The image processor 4, as shown in FIG. 2, has a DSC (Digital Scan Converter) 4A, a B-mode diagnostic image generator 4B, an M-mode diagnostic image generator 4C, and an image synthesizer 4D.

The DSC 4A, by performing a scan conversion process, converts the data output from the signal processor 3 into information expressed as Cartesian coordinates. The DSC 4A outputs the information converted into Cartesian coordinates to the B-mode diagnostic image generator 4B and the M-mode diagnostic image generator 4C.

The B-mode diagnostic image generator 4B generates B-mode diagnostic images based on the information converted into Cartesian coordinates, which has been output from the DSC 4A. The B-mode diagnostic image generator 4B outputs the B-mode diagnostic image data to the image synthesizer 4D.

The M-mode diagnostic image generator 4C generates optional-direction M-mode diagnostic images from the information converted into Cartesian coordinates, which has been output from the DSC 4A based on the optional-direction M-mode cursor set position information (described below), which has been output from the system controller 6. The M-mode diagnostic image generator 4C outputs the optional-direction M-mode diagnostic image data to the image synthesizer 4D.

The image synthesizer 4D synthesizes the B-mode diagnostic image data that has been output from the B-mode diagnostic image generator 4B, the M-mode diagnostic image data that has been output from the M-mode diagnostic image generator 4C, and the M-mode cursor image data (described below) that has been output from the system controller 6, and outputs the data after synthesis to a display 5.

The display 5 comprises, for example, a liquid crystal display.

The display 5 functions as a diagnostic image display part, displaying various kinds of operating screens and images, based on data that has been output from the image processor 4.

The system controller 6 comprises, for example, a CPU (Central Processing Unit), a ROM (Read Only Memory), a RAM (Random Access Memory) and an HDD (Hard Disk). The system controller 6 performs control of each unit by reading and executing programs, etc., that are stored in memory, based on input signals that have been input using the operation unit 7.

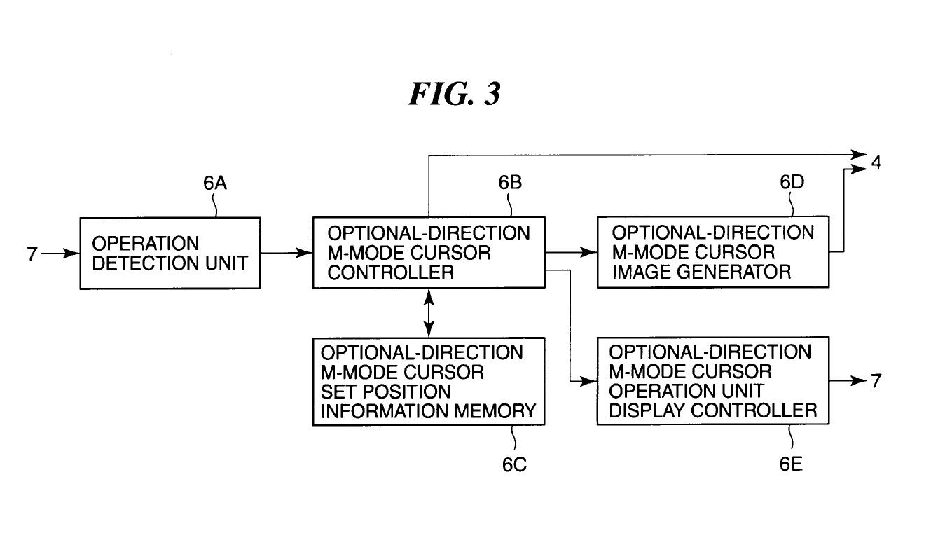

The system controller 6, as shown in FIG. 3, has an operation detection unit 6A, an optional-direction M-mode cursor controller 6B, an optional-direction M-mode cursor set position information memory 6C, an optional-direction M-mode cursor image generator 6D, and an optional-direction M-mode cursor operation unit display controller 6E.

The operation detection unit 6A functions as a detection means for detecting a prescribed operation that has been input by an operator, based on an input signal that has been input using the operation unit 7.

For example, when an operation is performed to instruct the display of various kinds of operating screens on the operation unit 7 (for example, display of the M-mode cursor selection icon for a diagnostic region), when an operation is performed to instruct the display of an image (for example, the M-mode cursor set operation), or when an operation is performed to switch from the B-mode display to the B/M-mode display, the operation detection unit 6A outputs a signal indicating the respective operation details to the optional-direction M-mode cursor controller 6B.

Furthermore, the operation detection unit 6A outputs to the optional-direction M-mode cursor controller 6B a signal indicating the operation details when an operation to select the optional-direction M-mode cursor is performed in operation unit 7.

The optional-direction M-mode cursor controller 6B controls the display of various kinds of operating screens and images on the display 5, based on signals indicating the operation details output from the operation detection unit 6A, controls the switching of the B-mode display screen being displayed on the display 5 to the B/M-mode display screen, etc.

The optional-direction M-mode cursor controller 6B reads the optional-direction M-mode cursor set position information, which is preset for each B-mode image scan cross-section, from the optional-direction M-mode cursor set position information memory 6C, based on a signal indicating the operation details that is output from the operation detection unit 6A. The M-mode cursor set position information that has been read is output to the optional-direction M-mode cursor image generator 6D, the optional-direction M-mode cursor operation unit display controller 6E, and the M-mode diagnostic image generator 4C of the image processor 4.

The optional-direction M-mode cursor set position information memory 6C functions as a memory part that stores the optional-direction M-mode cursor set position information (cursor center position, cursor length, cursor angle, etc.) that is preset for each scan cross-section of the B-mode image. For example, the scan cross-sections of the B-mode images for the left ventricle long axis view, Apical 4 Chamber view, Apical 2 Chamber view, Apical long axis view, and left ventricle short axis view are respectively associated, and the optional-direction M-mode cursor set position information is stored.

The optional-direction M-mode cursor image generator 6D generates data for displaying the optional-direction M-mode cursor image (lines), based on the optional-direction M-mode cursor set position information that has been output from the optional-direction M-mode cursor controller 6B, and outputs the optional-direction M-mode cursor image data to the image synthesizer 4D.

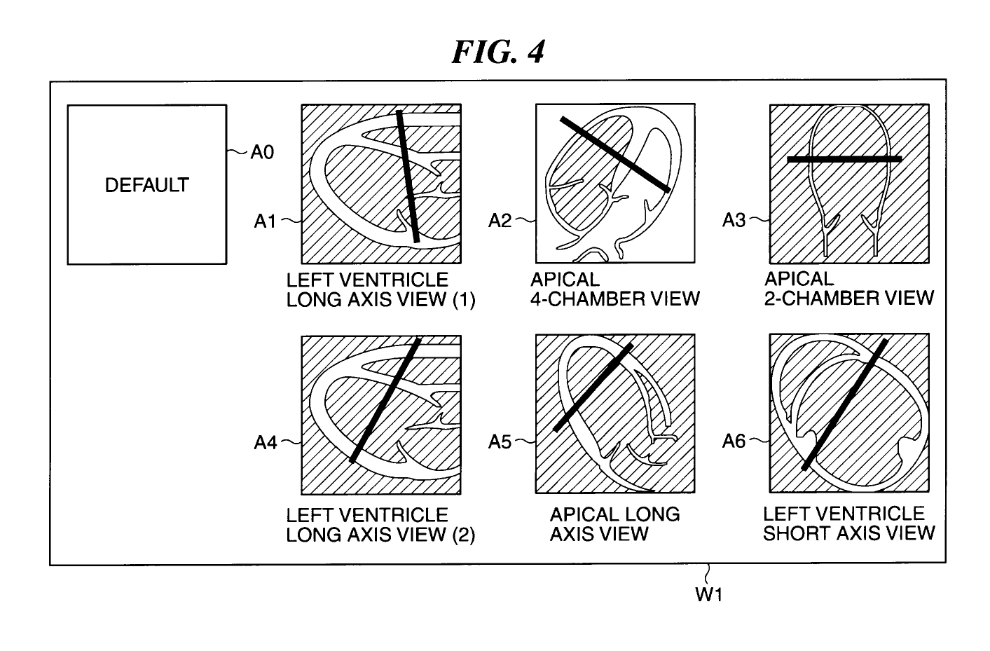

The optional-direction M-mode cursor operation unit display controller 6E displays on the Touch Command Screen of the operating unit 7 the selection icons (described below in FIG. 4), as the optional-direction M-mode cursor operation unit, based on the optional-direction M-mode cursor set position information that has been output from the optional-direction M-mode cursor controller 6B.

Furthermore, the optional-direction M-mode cursor operating unit display controller 6E superimposes the image of the optional-direction M-mode cursor that has been preset for each scan cross-section of the B-mode diagnostic image on the selection icons.

The operation unit 7 comprises an input device, such as a keyboard or a mouse, for the operator of an ultrasound diagnostic imaging device to input various kinds of operations, and a Touch Command Screen. The operation unit 7 generates an input signal to the system controller 6, based on the operations of the operator, and sends the input signal to the system controller 6. The Touch Command Screen is configured to enable the performing of operations by the operator directly pushing down the icons, etc. that are displayed on the screen.

The ultrasound diagnostic imaging device according to the present embodiment, if B/M simultaneous mode (simultaneous display of B-mode images and M-mode images) is selected by the operation unit 7, then together with displaying simultaneously on display 5 the B-mode diagnostic images and the M-mode diagnostic images, for example, as shown in FIG. 4, the cursor selection screen W1 for selecting the optional-direction M-mode cursor that is preset for each B-mode image scan cross-section is displayed on the Touch Command Screen.

In the display example of FIG. 4, icons A0 to A6 for selecting the optional-direction M-mode image cursor are arranged on the cursor selection screen W1. These icons A0 to A6 function as a selector for selecting the optional-direction M-mode image cursor.

The text “default” is displayed in icon A0. A morphological image representing a B-mode left ventricle long axis view and an optional-direction M-mode image cursor (the thick lines within the images in FIG. 4) for setting on that B-mode left ventricle long axis view are visually displayed in icon A1. A morphological image representing a B-mode apical 4-chamber view and an optional-direction M-mode image cursor for setting on that B-mode apical 4-chamber view are visually displayed in icon A2. A morphological image representing a B-mode apical 2-chamber view and an optional-direction M-mode image cursor for setting on that B-mode apical 2-chamber view are visually displayed in icon A3.

A morphological image representing a B-mode left ventricle long axis view and an optional-direction M-mode image cursor for setting on that B-mode left ventricle long axis view are visually displayed in icon A4. A morphological image representing a B-mode apical long axis view and an optional-direction M-mode image cursor for setting on that B-mode apical long axis view are visually displayed in icon A5. A morphological image representing a B-mode left ventricle short axis view and an optional-direction M-mode image cursor for setting on that B-mode left ventricle short axis view are visually displayed in icon A6.

The optional-direction M-mode cursor set position information for the B-mode diagnostic image scan cross-section that is stored in the optional-direction M-mode cursor set position information memory 6C is preset (associated) for these icons A0 to A6 respectively.

The optional-direction M-mode cursor set position information that is preset in icon A0 is default information (for example, information for forming a line connecting the irradiation position and depth of the ultrasound beam in an almost vertical direction), but the default settings may be made capable of modification by the operator.

Furthermore, the optional-direction M-mode cursor set position information that is preset in icons A1 to A6 may also be made capable of modification by the operator. Moreover, display position of the M-mode image cursor may also be made capable of modification by the operator.

The operator, by just pushing down on any of the icons A0 to A6 on the cursor selection screen W1 that is displayed on the Touch Command Screen, causes the optional-direction M-mode cursor set position information to be read from the optional-direction M-mode cursor set position information memory 6C, and can easily set the optional-direction M-mode cursor corresponding to the diagnostic region during an examination (corresponding to the position on the subject at which the ultrasound probe 1 is applied).

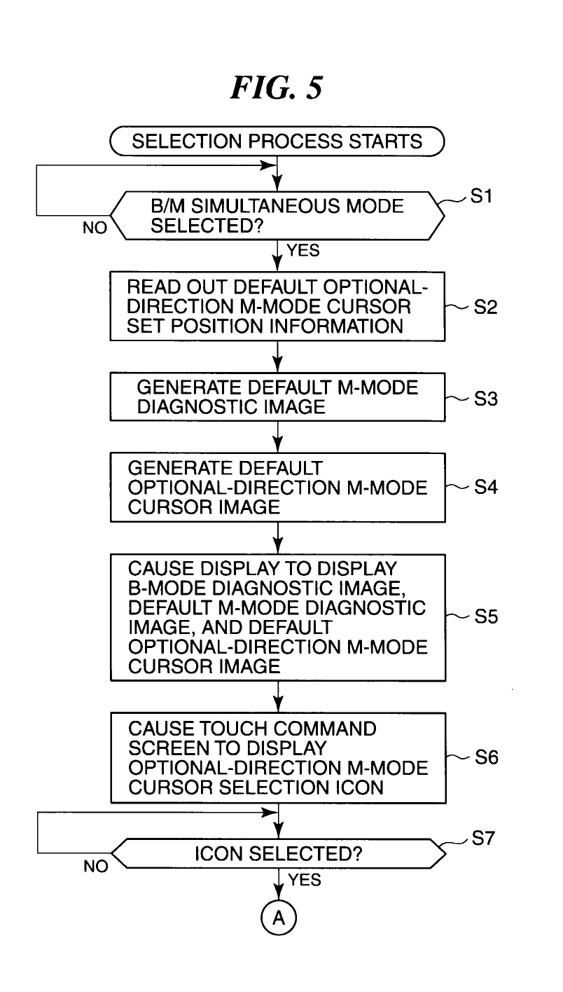

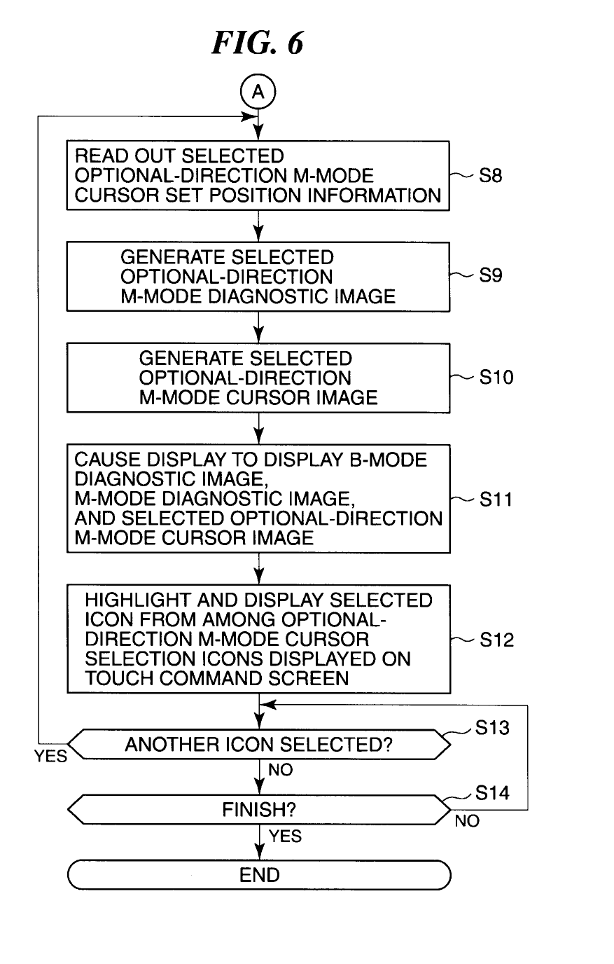

Next, referring to the flow charts in FIG. 5 and FIG. 6, the selection process for the optional-direction M-mode cursor displayed on the B-mode diagnostic image is described. When this process is started, the B-mode diagnostic image is displayed on the display 5.

In step S1, the operation detection unit 6A determines whether the B/M simultaneous mode has been selected in the operation unit 7 or not, and waits until the B/M simultaneous mode has been selected in the operation unit 7. Subsequently, in step S1, if the B/M simultaneous mode has been selected in the operation unit 7, the operation detection unit 6A outputs a signal indicating the details of that operation to the optional-direction M-mode cursor controller 6B. The processing proceeds to step S2.

In step S2, the optional-direction M-mode cursor controller 6B reads the default optional-direction M-mode cursor set position information from the optional-direction M-mode cursor set position information memory 6C, based on a signal indicating the details of the operation that has been output from the operation detection unit 6A.

The default optional-direction M-mode cursor set position information that has been read is output to the optional-direction M-mode cursor image generator 6D, the optional-direction M-mode cursor operation unit display controller 6E, and the M-mode diagnostic image generator 4C.

In step S3, the M-mode diagnostic image generator 4C generates a default M-mode diagnostic image, based on the default optional-direction M-mode cursor set position information that has been output from the optional-direction M-mode cursor controller 6B.

In Step S4, the optional-direction M-mode cursor image generator 6E generates a default optional-direction M-mode cursor image for synthesis on the B-mode diagnostic image, based on the default optional-direction M-mode cursor set position information that has been output from the optional-direction M-mode cursor controller 6B.

In step S5, the image synthesizer 4D synthesizes the B-mode diagnostic image data that has been generated by the B-mode diagnostic image generator 4B, the default M-mode diagnostic image data that has been generated by the M-mode diagnostic image generator 4D in the processing of step S3, and the default M-mode cursor image data that has been generated by the optional-direction M-mode cursor image generator 6E in the processing of step S4, and simultaneously displays them on the display 5.

In Step S6, the optional-direction M-mode cursor operation unit display controller 6E displays on the Touch Command Screen of the operating unit 7 the selection icons of the optional-direction M-mode cursor, based on the optional-direction M-mode cursor set position information that has been output from the optional-direction M-mode cursor controller 6B.

From this, as shown in FIG. 4, the cursor selection screen W1 (optional-direction M-mode cursor selection icons) for selecting the optional-direction M-mode cursor that is preset for each B-mode image scan cross-section is displayed on the Touch Command Screen of the operation unit 7. Furthermore, the optional-direction M-mode cursor images are synthesized on the selection icons, based on the optional-direction M-mode cursor set position information. In addition, icon A0 (default) is displayed highlighted.

In step S7, the operation detection unit 6A determines whether any of the icons A1 to A6 arranged on the cursor selection screen W1 of the Touch Command Screen have been pushed down or not, and waits until any of the icons A1 to A6 have been pushed down.

Subsequently, in step S7, if it is determined that any of the icons A1 to A6 have been pushed down, the operation detection unit 6A outputs a signal indicating the details of that operation to the optional-direction M-mode cursor controller 6B. The processing proceeds to step S8.

The processing from step S8 onward is described, referring to the flow chart in FIG. 6.

In step S8, the optional-direction M-mode cursor controller 6B reads the selected optional-direction M-mode cursor set position information from the optional-direction M-mode cursor set position information memory 6C, based on a signal indicating the operation details that is output from the operation detection unit 6A. The optional-direction M-mode cursor controller 6B outputs the selected optional-direction M-mode cursor set position information to the optional-direction M-mode cursor image generator 6D, the optional-direction M-mode cursor operation unit display controller 6E, and the M-mode diagnostic image generator 4C.

In step S9, the M-mode diagnostic image generator 4C generates M-mode diagnostic image in the selected optional-direction based on the optional-direction M-mode cursor set position information that has been output from the optional-direction M-mode cursor controller 6B.

In step S10, the optional-direction M-mode cursor image generator 6E generates the optional-direction M-mode cursor image for synthesis on the B-mode diagnostic image, based on the optional-direction M-mode cursor set position information that has been output from the optional-direction M-mode cursor controller 6B.

In step S11, the image synthesizer 4D synthesizes the B-mode diagnostic image data that has been generated by the B-mode diagnostic image generator 4B, the M-mode diagnostic image data that has been generated by the M-mode diagnostic image generator 4C in the processing of step S9, and the M-mode cursor image data that has been generated by the optional-direction M-mode cursor image generator 6E in the processing of step S10, and simultaneously displays them on the display 5.

In step S12, the optional-direction M-mode cursor operation unit display controller 6E displays in a highlighted state the icon selected in the processing of step S7, among optional-direction M-mode cursor selection icons being displayed on the Touch Command Screen of the operation unit 7.

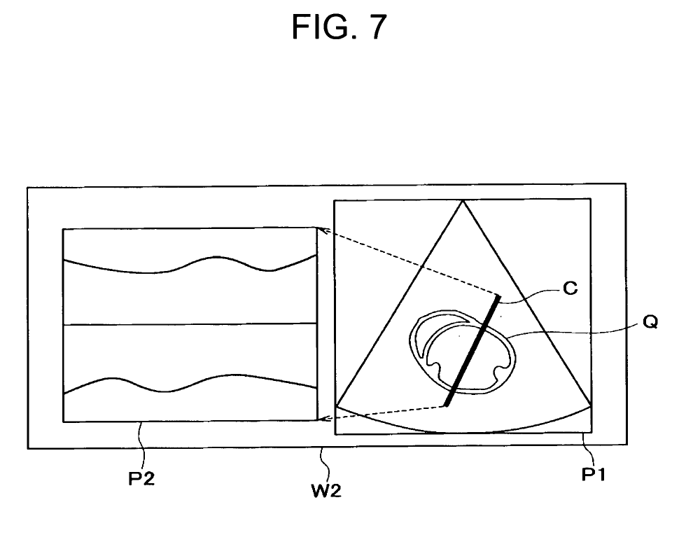

FIG. 7 is a figure showing a display example of a B/M-mode display screen W2 when an optional-direction M-mode cursor image is superimposed on a B-mode diagnostic image in B/M simultaneous mode.

As shown in FIG. 7, the B-mode diagnostic image P1, concerning the diagnostic region Q within the subject's body, is displayed in the B-mode diagnostic image display area on the right side of the screen in the B/M-mode display screen W2. On the B-mode diagnostic image P1, the optional-direction M-mode cursor image C is displayed. The optional-direction M-mode diagnostic image P2 for the optional-direction M-mode cursor image C of the B-mode diagnostic image is displayed in the M-mode diagnostic image display area on the left side of the screen.

In this way, by a simple operation of selecting any of the icons A1 to A6 arranged on the cursor selection screen W1 that is displayed on the Touch Command Screen, the optional-direction M-mode cursor image C can be set on the B-mode diagnostic image P1.

In step S13, the operation detection unit 6A determines whether any of the icons A1 to A6 arranged on the cursor selection screen W1 of the Touch Command Screen have been pushed down or not. In other words, the operation detection unit 6A, after the position of applying the ultrasound probe 1 is modified by the operator, determines whether any of the icons A1 to A6 arranged on the cursor selection screen W1 of the Touch Command Screen have been pushed down or not.

In step S13, if the operation detection unit 6A determines that any icon among the icons A1 to A6 has been selected (step S13, YES), then processing returns to step S8, and the abovementioned processing is performed again. In this way, optional-direction M-mode cursor set position information based on a newly selected scan cross-section is read (step S8), optional-direction M-mode diagnostic images are generated for the newly selected scan cross-section (step S9), optional-direction M-mode cursor images are generated for the newly selected scan cross-section (step S10), and the B-mode diagnostic images, M-mode diagnostic images and optional-direction M-mode cursor images are synthesized and displayed (step S11).

In Step S13, if none of the icons among the icons A1 to A6 has been selected (step S13, NO), the processing proceeds to step S14.

Furthermore, the operation detection unit 6A determines whether an operation has been performed to instruct examination termination or display termination in operation unit 7, and if it determines that it is not examination termination or display termination, then it returns to step S13, and the abovementioned processing is performed again.

In step S14, if it is determined that it is examination termination or display termination, then the selection process of the optional-direction M-mode cursor is terminated.

As described above, the operator, while applying the ultrasound probe 1 to the subject, by a simple operation of pushing down once on an icon displayed on the Touch Command Screen, can set the optional-direction M-mode cursor (center position, length, angle) on the B-mode diagnostic image.

Moreover, in cases where the scan cross-section is modified also, by a simple operation of pushing down once on an icon displayed on the Touch Command Screen, the optional-direction M-mode cursor can be set again. Hence, along with reducing the burden on the operator, it is possible to achieve an improvement in examination efficiency and a reduction in examination time.

In the above description, a selector for selecting an optional-direction M-mode cursor for a scan cross-section of a B-mode diagnostic image is implemented by displaying icons on the Touch Command Screen. In the present embodiment, without being limited to icons, for example, in the operation unit 7, a button or switch functioning as an optional-direction M-mode cursor selector may be provided.

### (When a Button Functions as an Optional-Direction M-Mode Cursor Selector)

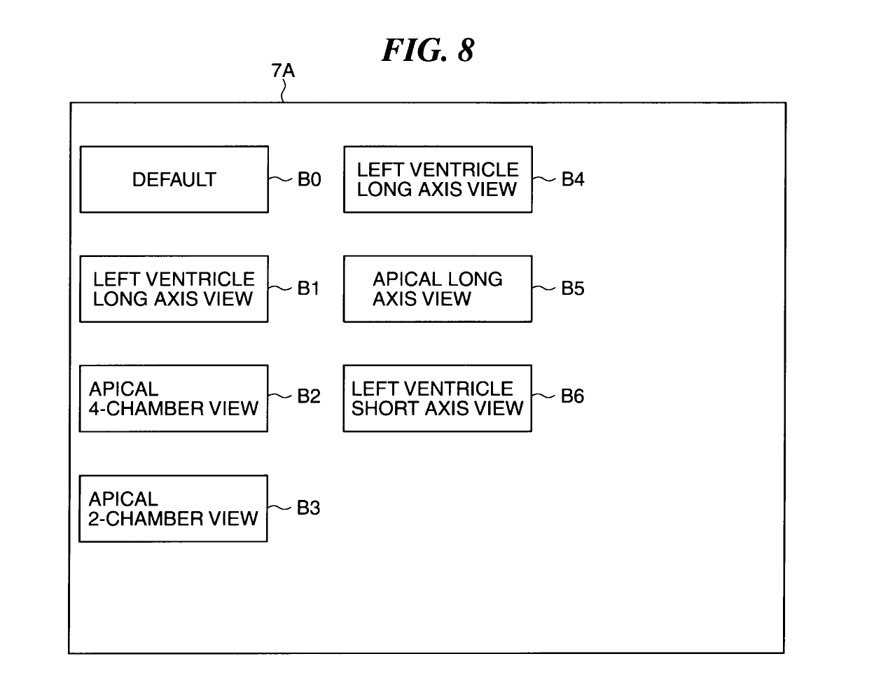

Referring to FIG. 8, one example of a button functioning as an optional-direction M-mode cursor selector is described. FIG. 8 is a figure showing a console in which buttons are installed.

In a console 7A of operation unit 7, buttons B0 to B6 for selecting the optional-direction M-mode image cursor have been arranged. These buttons B0 to B6 function as a selector for selecting the optional-direction M-mode image cursor. Text indicating the respective scan cross-sections is attached to buttons B0 to B6.

For example, the text “default” is attached to button B0.

The text “left ventricle long axis view 1” in B-mode is attached to button B1.

The text “Apical 4 Chamber view” in B-mode is attached to button B2.

The text “Apical 2 Chamber view” in B-mode is attached to button B3.

The text “left ventricle long axis view 2” in B-mode is attached to button B4.

The text “Apical long axis view” in B-mode is attached to button B5.

The text “left ventricle short axis view” in B-mode is attached to button B6.

For each of the buttons B0 to B6, the optional-direction M-mode cursor set position information for the scan cross-section of the B-mode diagnostic image, which is stored in the optional-direction M-mode cursor set position information memory 6C, is preset (associated).

The optional-direction M-mode cursor set position information that is preset for button B0 is the default information (for example, information for forming a line connecting the irradiation position and depth of the ultrasound beam in an almost vertical direction), but the default settings may be made capable of modification by the operator.

Furthermore, the optional-direction M-mode cursor set position information that is preset in buttons B1 to B6 may also be made capable of modification by the operator.

The operator, by just pushing down on any of the buttons B0 to B6, causes the optional-direction M-mode cursor set position information to be read from the optional-direction M-mode cursor set position information memory 6C, and can easily set the optional-direction M-mode cursor corresponding to the diagnostic region during an examination.

### (When a Switch Functions as the Optional-Direction M-Mode Cursor Selector)

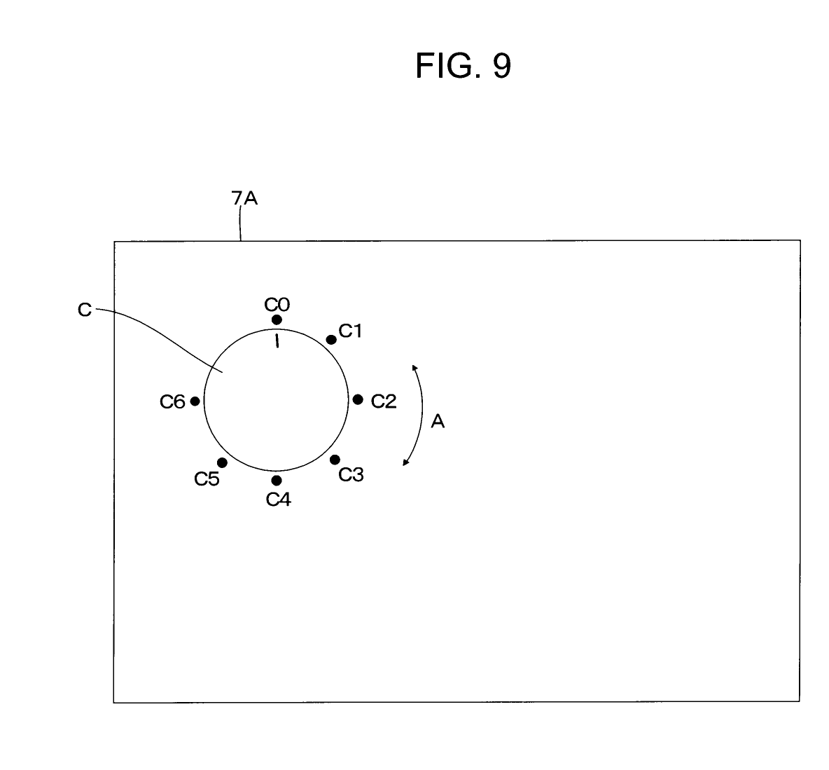

Next, referring to FIG. 9, one example of a switch functioning as an optional-direction M-mode cursor selector is described. FIG. 9 is a figure showing a console in which a switch is installed. As one example of a switch, a rotary switch is described.

In a console 7A of operation unit 7, rotary switch C is provided for selecting the optional-direction M-mode image cursor. This rotary switch C functions as a selector for selecting the optional-direction M-mode image cursor. The rotary switch C is configured to be rotatable in the direction of the arrow A.

Although it is not shown in FIG. 9, text indicating the scan cross-sections is attached, at prescribed angles, spaced apart, in the rotational direction A of the rotary switch C.

For example, the text “default” is attached to the position of angle C0.

The text “left ventricle long axis view 1” in B-mode is attached to the position of angle C1.

The text “Apical 4 Chamber view” in B-mode is attached to the position of angle C2.

The text “Apical 2 Chamber view” in B-mode is attached to the position of angle C3.

The text “left ventricle long axis view 2” in B-mode is attached to the position of angle C4.

The text “Apical long axis view” in B-mode is attached to the position of angle C5.

The text “left ventricle short axis view” in B-mode is attached to the position of angle C6.

For each of the angles C0 to C6, the optional-direction M-mode cursor set position information for the scan cross-section of the B-mode diagnostic image, which is stored in the optional-direction M-mode cursor set position information memory 6C, is preset (associated).

The optional-direction M-mode cursor set position information that is preset for angle C0 is the default information (for example, information for forming a line connecting the irradiation position and depth of the ultrasound beam in an almost vertical direction), but the default settings may be made capable of modification by the operator.

Furthermore, the optional-direction M-mode cursor set position information that is preset in angles C1 to C6 may also be made capable of modification by the operator.

The operator, by just rotating the rotary switch C and selecting any of the angles C0 to C6, causes the optional-direction M-mode cursor set position information to be read from the optional-direction M-mode cursor set position information memory 6C, and can easily set the optional-direction M-mode cursor corresponding to the diagnostic region during an examination.

### (When a Menu Functions as the Optional-Direction M-Mode Cursor Selector)

Moreover, a menu functioning as an optional-direction M-mode cursor selector may be displayed on display 5.

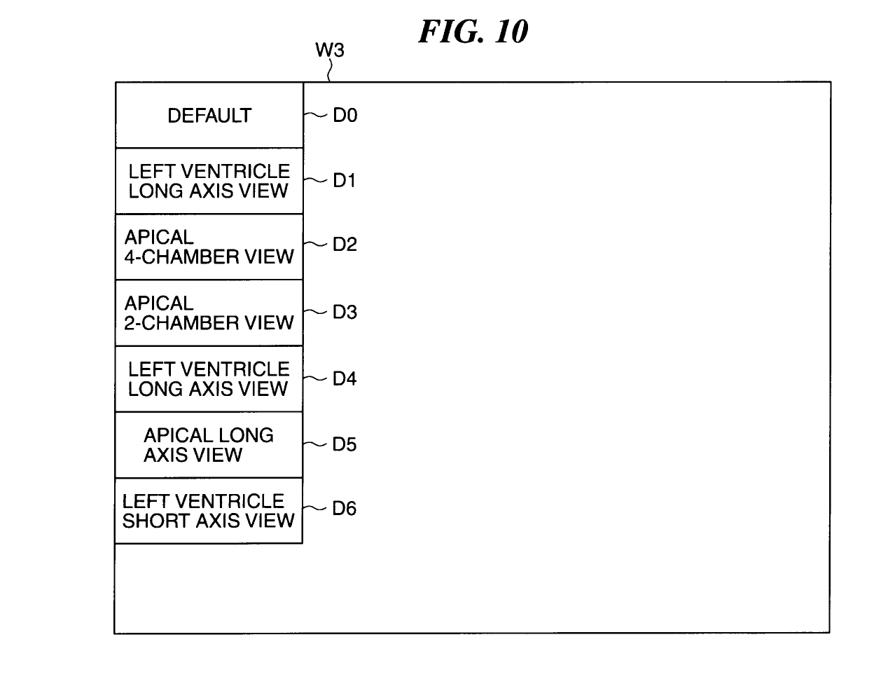

Next, referring to FIG. 10, one example of a menu functioning as an optional-direction M-mode cursor selector is described. FIG. 10 is an example of a display screen W3 on which menus are displayed.

Display 5 displays as a list menu D0 to D6, for selecting the optional-direction M-mode image cursor. These menus D0 to D6 function as a selector for selecting the optional-direction M-mode image cursor. Text indicating the respective scan cross-sections is shown on menus D0 to D6.

For example, the text “default” is displayed on menu D0.

The text “left ventricle long axis view 1” in B-mode is displayed on menu D1.

The text “Apical 4 Chamber view” in B-mode is displayed on menu D2.

The text “Apical 2 Chamber view” in B-mode is displayed on menu D3.

The text “left ventricle long axis view 2” in B-mode is displayed on menu D4.

The text “Apical long axis view” in B-mode is displayed on menu D5.

The text “left ventricle short axis view” in B-mode is displayed on menu D6.

For each of the menus D0 to D6, the optional-direction M-mode cursor set position information for the scan cross-section of the B-mode diagnostic image, which is stored in the optional-direction M-mode cursor set position information memory 6C, is preset (associated).

The optional-direction M-mode cursor set position information that is preset for menu D0 is the default information (for example, information for forming a line connecting the irradiation position and depth of the ultrasound beam in an almost vertical direction), but the default settings may be made capable of modification by the operator.

Furthermore, the optional-direction M-mode cursor set position information that is preset in menus D1 to D6 may also be made capable of modification by the operator.

The operator, by just selecting any of the menus D0 to D6 that are displayed on display 5 using operation unit 7, causes the optional-direction M-mode cursor set position information to be read from the optional-direction M-mode cursor set position information memory 6C, and can easily set the optional-direction M-mode cursor corresponding to the diagnostic region during an examination.

### (When the Optional-Direction M-Mode Cursor is Automatically Set)

Moreover, the optional-direction M-mode cursor for a scan cross-section of a B-mode diagnostic image may be automatically set.

As one example, the optional-direction M-mode cursor controller 6B may function as a selector for selecting the optional-direction M-mode cursor.

For example, the optional-direction M-mode cursor controller 6B receives B-mode diagnostic image data output from the B-mode diagnostic image generator 4B. The optional-direction M-mode cursor controller 6B, by performing image recognition by pattern matching against the B-mode diagnostic image data, identifies the scan cross-section scanned by ultrasound from the B-mode diagnostic image data.

For example, if the image displayed in the B-mode diagnostic image is a left ventricle long axis view, the optional-direction M-mode cursor controller 6B identifies that the scan cross-section scanned by ultrasound is a left ventricle long axis view scan cross-section.

Moreover, if the image displayed in the B-mode diagnostic image is an Apical 4 Chamber view, the optional-direction M-mode cursor controller 6B identifies that the scan cross-section scanned by ultrasound is an Apical 4 Chamber view scan cross-section. Moreover, if the image displayed in the B-mode diagnostic image is an Apical 2 Chamber view, the optional-direction M-mode cursor controller 6B identifies that the scan cross-section scanned by ultrasound is an Apical 2 Chamber view scan cross-section. Moreover, if the image displayed in the B-mode diagnostic image is an Apical long axis view, the optional-direction M-mode cursor controller 6B identifies that the scan cross-section scanned by ultrasound is an Apical long axis view scan cross-section. Moreover, if the image displayed in the B-mode diagnostic image is a left ventricle short axis view, the optional-direction M-mode cursor controller 6B identifies that the scan cross-section scanned by ultrasound is a left ventricle short axis view scan cross-section.

The optional-direction M-mode cursor controller 6B reads the optional-direction M-mode cursor set position information associated with the identified scan cross-section from the optional-direction M-mode cursor set position information memory 6C. The optional-direction M-mode cursor set position information that is read is output to the optional-direction M-mode cursor image generator 6D, the optional-direction M-mode cursor operation unit display controller 6E, and the M-mode diagnostic image generator 4C of the image processor 4.

The optional-direction M-mode cursor image generator 6D generates data for displaying the optional-direction M-mode cursor image (lines), based on the optional-direction M-mode cursor set position information that is output from the optional-direction M-mode cursor controller 6B, and outputs the optional-direction M-mode cursor image data to the image synthesizer 44.

The image synthesizer 4D synthesizes the B-mode diagnostic image generated by the B-mode diagnostic image generator 4B, the M-mode diagnostic image generated by the M-mode diagnostic image generator 4D, and the optional-direction M-mode cursor image generated by the optional-direction M-mode cursor image generator 6E, and simultaneously displays them on the display 5.

As described above, by identifying the scan cross-section using pattern matching, it is possible to automatically set the optional-direction M-mode cursor on the B-mode diagnostic image.

Moreover, if the scan cross-section is modified, it is possible to automatically set the optional-direction M-mode cursor. Hence, along with reducing the burden on the operator, it is possible to achieve an improvement in examination efficiency and a reduction in examination time.

While certain embodiments have been described, these embodiments have been presented by way of example only, and are not intended to limit the scope of the inventions. Indeed, the novel methods and devices described herein may be embodied in a variety of other forms; furthermore, various omissions, substitutions and changes in the form of the methods and devices described herein may be made without departing from the spirit of the inventions. The accompanying claims and their equivalents are intended to cover such forms or modifications as would fall within the scope and spirit of the inventions.

What is claimed is:

1. An ultrasound diagnostic imaging device, comprising:
a B-mode diagnostic image generator that generates B-mode diagnostic images based on echo signals obtained by scanning a subject using ultrasound;
an optional-direction M-mode diagnostic image generator that generates optional-direction M-mode diagnostic images based on set position information of an optional-direction M-mode cursor;
an optional-direction M-mode cursor set position information selector for selecting said optional-direction M-mode cursor set position information;
a memory for storing said optional-direction M-mode cursor set position information, associating it with said optional-direction M-mode cursor set position information selector; and
an optional-direction M-mode cursor image generator that generates an optional-direction M-mode cursor image, based on said optional-direction M-mode cursor set position information that has been associated with said optional-direction M-mode cursor set position information selector that was selected.

2. The ultrasound diagnostic imaging device of claim 1, further comprising a diagnostic image display that, along with superimposing said optional-direction M-mode cursor image that was generated by said optional-direction M-mode cursor image generator on said B-mode diagnostic images that were generated by said B-mode diagnostic image generator, displays said optional-direction M-mode diagnostic image that was generated by said optional-direction M-mode diagnostic image generator.

3. The ultrasound diagnostic imaging device of claim 1, wherein said optional-direction M-mode cursor set position information selector is an icon displayed on a Touch Command Screen, a switch or a button provided in an operation unit.

4. The ultrasound diagnostic imaging device of claim 3, wherein B-mode morphological images showing the diagnostic regions, and an optional-direction M-mode cursor for setting on said B-mode morphological images are visually displayed on said icons.

5. The ultrasound diagnostic imaging device of claim 4, wherein said B-mode morphological image is a cardiac left ventricle long axis view, Apical 4 Chamber view, Apical 2 Chamber view, Apical long axis view, or left ventricle short axis view.

6. The ultrasound diagnostic imaging device of claim 3, wherein the selected said icon is displayed highlighted.

7. The ultrasound diagnostic imaging device of claim 1, wherein said optional-direction M-mode cursor set position information comprises at least the center position, length and angle of said optional-direction M-mode cursor.

8. The ultrasound diagnostic imaging device of claim 1, wherein:
said optional-direction M-mode cursor set position information selector is a menu displayed on a display;
said memory stores said optional-direction M-mode cursor set position information, associating it with said menu; and
said optional-direction M-mode cursor image generator generates said optional-direction M-mode cursor image, based on said optional-direction M-mode cursor set position information that has been associated with the selected said menu.

9. The ultrasound diagnostic imaging device of claim 1, wherein:
said memory stores said optional-direction M-mode cursor set position information, associating it with a scan cross-section identified by said optional-direction M-mode cursor set position information selector; and
said optional-direction M-mode cursor image generator generates said optional-direction M-mode cursor image, based on said optional-direction M-mode cursor set position information that has been associated with a scan cross-section identified by the selected said optional-direction M-mode cursor set position information selector.

10. The ultrasound diagnostic imaging device of claim 9, wherein said scan cross-section is a cross-section for said B-mode diagnostic image generator to obtain, by ultrasound scan, said B-mode diagnostic images showing a cardiac left ventricle long axis view, Apical 4 Chamber view, Apical 2 Chamber view, Apical long axis view, or left ventricle short axis view.

11. The ultrasound diagnostic imaging device of claim 1, wherein said optional-direction M-mode cursor set position information selector is used for selecting, said optional-direction M-mode cursor set position information.

12. The ultrasound diagnostic imaging device of claim 1, wherein:
said optional-direction M-mode cursor set position information selector identifies, based on said B-mode diagnostic images, a scan cross-section that has been scanned using ultrasound;
said memory stores said optional-direction M-mode cursor set position information, associating it with a scan cross-section identified by said optional-direction M-mode cursor set position information selector; and
said optional-direction M-mode cursor image generator generates said optional-direction M-mode cursor image based on said optional-direction M-mode cursor set position information that has been associated with said scan cross-section, which was identified by said optional-direction M-mode cursor set position information selector.

13. An ultrasound image generation method that:
generates B-mode diagnostic images based on echo signals obtained by scanning a subject using ultrasound;
generates optional-direction M-mode diagnostic images based on set position information of an optional-direction M-mode cursor; and
when said optional-direction M-mode cursor set position information selector for selecting optional-direction M-mode cursor set position information is selected, identifies said optional-direction M-mode cursor set position information that was associated with the selected said optional-direction M-mode cursor set position information selector, in accordance with the association between said optional-direction M-mode cursor set position information selector and said optional-direction M-mode cursor set position information, and generates an optional-direction M-mode cursor image, based on said optional-direction M-mode cursor set position information that was identified.

14. The ultrasound image generation method of claim 13 that along with superimposing said optional-direction M-mode cursor image that was generated on said B-mode diagnostic images, displays said optional-direction M-mode diagnostic images.

15. The ultrasound image generation method of claim 13, wherein said optional-direction M-mode cursor set position information selector is an icon displayed on a Touch Command Screen, a switch or a button provided in an operation unit.

16. The ultrasound image generation method of claim 15, wherein B-mode morphological images showing the diagnostic regions, and an optional-direction M-mode cursor for setting on said B-mode morphological image are visually displayed on said icons.

17. The ultrasound image generation method of claim 16, wherein said B-mode morphological image is a cardiac left ventricle long axis view, Apical 4 Chamber view, Apical 2 Chamber view, Apical long axis view, or left ventricle short axis view.

18. The ultrasound image generation method of claim 15, wherein the selected said icon is displayed highlighted.

19. The ultrasound image generation method of claim 13, wherein said optional-direction M-mode cursor set position information comprises at least the center position, length and angle of said optional-direction M-mode cursor.

20. The ultrasound image generation method of claim 13, wherein:
said optional-direction M-mode cursor set position information selector identifies, based on said B-mode diagnostic images, a scan cross-section that has been scanned using ultrasound; and
said optional-direction M-mode cursor image is generated based on said optional-direction M-mode cursor set position information that has been associated with said scan cross-section, which was identified by said optional-direction M-mode cursor set position information selector.
